(19) United States
(12) Patent Application Publication
(10) Pub. No.: US 2013/0110235 A1
(43) Pub. Date: May 2, 2013

(54) ACCOMMODATING INTRAOCULAR LENS WITH DEFORMABLE MATERIAL

(71) Applicant: James T. Schwiegerling, Tucson, AZ (US)

(72) Inventor: James T. Schwiegerling, Tucson, AZ (US)

(73) Assignee: The Arizona Board of Regents on Behalf of the University of Arizona, Tucson, AZ (US)

(21) Appl. No.: 13/806,993

(22) PCT Filed: Jun. 29, 2011
(86) PCT No.: PCT/US11/42446

§ 371 Date: Dec. 26, 2012

Related U.S. Application Data
(60) Provisional application No. 61/398,626, filed on Jun. 29, 2010.

(51) Int. Cl. A61F 2/16

(52) U.S. Cl. CPC A61F 2/16
USPC 623/6.56

(57) ABSTRACT

An accommodating intraocular lens. The lens includes a substantially-rigid anterior member having an extrusion aperture. First transparent deformable material is disposed anterior to the posterior side of the anterior member. Second transparent deformable material is disposed adjacent the posterior surface of the first material, the second material having a different degree of deformability than the first material and having an index of refraction different from the index of refraction of the first material. This forms a refractive deformable interface between the body of first material and the body of the second material. Force applied to the second material causes that material to be extruded through the aperture so as to form a curved, refractive interface with the body of first material. A method for installation of the accommodating intraocular lens is also provided.

### RELATED APPLICATIONS

This patent application claims the benefit of U.S. provisional patent application No. 61/398,626, filed on Jun. 29, 2011, which is hereby incorporated by reference in its entirety.

### TECHNICAL FIELD

The present invention relates to intraocular lenses used in ophthalmology, and more specifically, to the structures and methods of use of accommodating intraocular lenses to for the treatment of presbyopia.

### BACKGROUND

Presbyopia is a visual disorder in which the ability of the eye to accommodate decreases with age. This loss of accommodation is present in youth, but the amplitude of accommodation remains sufficiently high that the effects of presbyopia are largely ignored. In the fifth decade of human life, however, the loss of accommodation typically reaches a point where full exertion is needed to bring near objects into focus. This strain leads to rapid fatigue. Furthermore, the loss continues and eventually the amplitude of accommodation is minimal, leading to an eye normally corrected for distance being incapable of focusing on near objects. Consequently, presbyopia affects an individual's ability to read, view a computer monitor and perform other near tasks.

External appliances such as reading glasses and bifocal and progressive addition spectacle lenses have routinely been used to overcome the presbyope's lack of accommodation. These glasses at the very least require the presbyope to be dependent upon an external device for good vision and these devices are subject to be lost or broken, as well as require periodic testing to ensure the prescription is still valid. Furthermore, the bifocal and progressive type lenses suffer from additional issues such as distortion in the magnification of objects (e.g. image jump), artifacts in peripheral vision known as swim, and a significant effort in adaptation to their use. Implanted devices such as accommodating intraocular lenses (IOLs) have also been described to treat presbyopia. One example is described by J. Ben-nun and J. L. Alio, “Feasibility and development of a high power real accommodating intraocular lens.” J Cataract Ref Surg 2005; 31:1802-1808, hereby incorporated by reference in its entirety and referred to herein as “Ben-nun.”

Presbyopia is also treated with multifocal contact lenses and IOLs. The multifocal effect is typically achieved by employing refractive or diffractive elements to embed multiple powers into a single lens. The multifocal effect gives simultaneous vision in which both in-focus and out-of-focus images are superimposed on the retina. The distance portion of the lens provides sharp distance vision and blurry near vision. The near portion of the lens provides sharp near vision and blurry distance vision. The presbyope must learn to ignore the blurry information and interpret the sharp information. In general, multifocal lenses result in a visual compromise in which two discrete planes are in focus (as opposed to the continuous range in an eye with accommodation), and a degradation in contrast occurs at both these planes due to the superposition of sharp and blurry images.

An IOL is an artificial replacement lens that may be used as an alternative to a contact lens or eyeglasses. An IOL is often implanted in place of the natural eye lens during cataract surgery. To overcome the limitations of existing presbyopia treatments, accommodating IOLs have been aggressively pursued in recent years. The ideal accommodating IOL would act much like the young crystalline lens in that it should provide a large range of accommodation in response to ciliary muscle contraction. The optical performance of the ideal lens should also give sharp, high contrast images over the accommodative range. Existing accommodating IOL technologies fall far short of this ideal lens performance.

To allow focusing from distance to 33 cm, an accommodating IOL should provide a minimum 3 diopters (D) of accommodation. The total power of the eye, Φ_{eye}, is given by

$${Φ}_{eye} = {φ}_{cor} + {φ}_{lens} - \frac{t}{{n}_{aq}} {φ}_{cor} \cdot {φ}_{lens} , \qquad ( 1 )$$

where φ_{cor} is the power of the cornea, φₗₑₙₛ is the power of the lens (either crystalline lens or IOL), t is the separation between the cornea and lens and n_{aq} is the refractive index of the aqueous. To focus on near objects, an increase in the ocular power is needed. One way of achieving this change is by changing the separation, t, between the cornea and lens. To determine the effect of this change, eq. 1 can be differentiated with respect to t.

$${ΔΦ}_{eye} = - \frac{Δ t}{{n}_{aq}} {φ}_{cor} \cdot {φ}_{lens} ⇒ Δ t = - \frac{{n}_{aq} {ΔΦ}_{eye}}{{φ}_{cor} \cdot {φ}_{lens}} \qquad ( 2 )$$

Eq. 2 suggests that to get ΔΦ_{eye}=3 D of accommodation, Δt=−4.6 mm for φ_{cor}=43 D, φₗₑₙₛ=20 D and n_{aq}=1.336. In other words, an axially translating single optic IOL would need to nearly press against the posterior cornea to achieve 3 D of accommodation. Physical limitations on the placement of the lens in the eye, interference with the iris and limited movement of the ciliary muscle make this technique horribly inefficient at providing accommodation.

A second way of achieving accommodation is to change the power of the lens. Again, eq. 1 can be differentiated this time with respect to φₗₑₙₛ. In this case,

$${ΔΦ}_{eye} = [ 1 - \frac{t}{{n}_{aq}} {φ}_{cor} ] \cdot {Δφ}_{lens} ⇒ {ΔΦ}_{eye} ≅ 0.87 {Δφ}_{lens} . \qquad ( 3 )$$

Eq. 3 shows that the change in the power of the eye is nearly proportional to the change in the power of the lens. One technique to achieve this power change in the lens is to construct a dual optic accommodating IOL in which the separation between the two lenses is changed with ciliary muscle contraction. Through similar arguments as eq. 2, the separation change is inefficient in providing the required power change. Alternatively, a curvature change in one or both surfaces of the lens can provide a power change. Assuming a thin lens,

φₗₑₙₛ=(nₗₑₙₛ−n_{aq})(c₁−c₂) (4)

where nₗₑₙₛ is the refractive index of the lens, and c₁ and c₂ are the anterior and posterior curvatures of the lens. While either or both curvatures could be changed to induce a change in lens power, for this analysis the anterior surface curvature c₁ will be assumed to be variable. Differentiating Eq. 4 with respect to c₁ gives

Δφₗₑₙₛ=(nₗₑₙₛ−n_{aq})Δc₁ .(5)

From eq. 3, to get ΔΦ_{eye}=3 D of accommodation, ΔΦₗₑₙ needs to be 3.4 D. Furthermore, using eq. 5 and assuming nₗₑₙₛ=1.5, the change in curvature Δc₁ needed to achieve this level of accommodation is 20.7 m⁻¹. A typical curvature of an IOL surface is 66.6 m⁻¹. Changing the surface curvature from 66.6 m⁻¹ to 66.6+20.7=87.3 m⁻¹ requires only a 93 micron change in sagittal depth of the surface over a 6 mm optical zone. In other words, an accommodating IOL in which the posterior surface and the edges of the anterior surface are fixed, but the anterior surface can be deformed to increase the center thickness of the lens by 93 microns would provide 3 D of accommodation. Thus, small changes in curvature can provide large changes in accommodation.

The first generation of accommodating IOLs operates by axially translating a single or dual optic within the eye. The translation provides an overall ocular power change, but the required magnitude of translation is enormous compared to the movement provided by the ciliary muscle. Consequently, these translation-based technologies have demonstrated little or no benefit to the presbyope. A. L. Sheppard, “Accommodating intraocular lenses: a review of design concepts, usage and assessment methods.” Clinical and Experimental Optometry 93.6 Nov. 2010, pp. 441-452.

Next generation accommodating IOLs use changes in surface curvature to produce accommodation. Substantial power changes are achieved with small curvature changes. This next generation of lenses shows promise to better treatment of presbyopia.

Curvature-changing accommodating IOLs have been demonstrated. One example is the FluidVision lens. A. L. Sheppard, supra. This lens has bladders that serve as reservoirs for fluid. As the ciliary muscle contracts, the bladders are compressed, thereby pumping fluid into the lens interior. The anterior surface of this lens is a membrane that deforms from the increased volume of fluid. In general, this leads to a fairly large lens.

A smaller alternative to this lens is a type of accommodating IOL shown schematically in FIG. 1, corresponding to the fully accommodated state of the human eye, and FIG. 2, corresponding to the fully unaccommodated state of the human eye. As is commonly known in the art, the natural lens of the eye is removed and the IOL is installed in its place. For an accommodating IOL, the anterior portion of the lens capsule removed or is collapsed so that the posterior portion of the IOL rests against the posterior portion of the lens capsule so as to operate the IOL in response to actuation of the ciliary muscles. This prior art lens 10 is referred to as the “NuLens IOL” and illustrated in FIGS. 1 and 2 in relation to the cornea 12 of the eye.

As shown in FIG. 1, the NuLens IOL lens employs a soft, elastic polymer 14 such as a hydrogel sandwiched between two rigid plates, that is, an anterior plate 16 and a posterior plate 18. The anterior plate is fixated within the eye so that it cannot move. The anterior plate also has a small aperture 20. When a compressive force, shown by arrows 22 in FIG. 22, is applied to the soft polymer 14 through the posterior plate 18, the polymer partially extrudes through the aperture 20, creating a curved surface 24. The added power of this surface is given by Δφₗₑₙₛ=(n_{aq}−n_{hydrogel})/R, where n_{hydrogel} is the refractive index of the soft polymer. When the compressive force is released, as shown in FIG. 1, the added power is eliminated, the amount of added power being determined by the amount of compressive force that is applied. Light rays 28 in FIG. 2 show the effect on focal length of the added power and light rays 26 in FIG. 1 show how the focal length changes in the absence of the added power. The focal length of the eye decreases when the compressive force is applied to the posterior plate. A physical embodiment of this principle is disclosed and described more fully in J. Ben-nun, supra.

However, this prior art accommodating IOL has a significant drawback. When the eye is in its unaccommodated state corresponding to FIG. 2, the ciliary muscle dilates and the posterior portion of the capsule is stretched. This stretching causes a compressive force to be applied to the NuLens IOL. When the eye accommodates corresponding to FIG. 1, the ciliary muscle constricts, releasing the tension on the capsule and thereby removing the compressive force on the IOL. In other words, the Nulens IOL is in its compressed state with added power when the eye is unaccommodated and the added power disappears when the eye accommodates.

This reversed mechanism of action is believed to cause problems with the use of this lens because there are two other physiological mechanisms, convergence and pupil miosis that occur in conjunction with accommodation.

Accordingly, there is a need for an accommodating IOL structure and method of use that provides accommodation that corresponds to the normal relationship between the amount of accommodation needed and the muscular movement of the ciliary body.

### SUMMARY

An accommodating intraocular lens and methods for providing an eye with an accommodating intraocular lens are disclosed. Preferably the lens includes a substantially-rigid anterior member having an anterior side and a posterior side and an extrusion aperture there through. A body of a first transparent deformable material is disposed anterior to the posterior side of the anterior member at least partially within the periphery of the extrusion aperture. A body of a second transparent deformable material having an anterior surface is disposed at least partially adjacent the posterior surface of the body of first transparent material, the second transparent deformable material having a different degree of deformability than the first transparent deformable material and having an index of refraction different from the index of refraction of the first transparent deformable material, thereby forming a refractive deformable interface between the body of first transparent deformable material and the body of second transparent deformable material. A posterior member having an anterior side and a posterior side, the anterior side being disposed against the posterior of the body of second transparent elastic material, such that upon application of force against the posterior side of the posterior member with the body of first transparent deformable material restrained so as to cause movement of the posterior member relative to the body of first transparent deformable material, a portion of the second transparent deformable material is extruded through the extrusion aperture so as to form a curved, refractive interface with the body of first transparent deformable material.

Preferably the method includes providing an accommodating intraocular lens having a substantially-rigid anterior member having an anterior side and a posterior side and an extrusion aperture there through; a body of a first transparent deformable material disposed anterior to the

posterior side of the anterior member at least partially within the periphery of the extrusion aperture; a body of a second transparent deformable material having an anterior surface disposed at least partially adjacent the posterior surface of the body of first transparent material, the second transparent deformable material having a different degree of deformability than the first transparent deformable material and having an index of refraction different from

the index of refraction of the first transparent deformable material, thereby forming a refractive deformable interface between the body of first transparent deformable material and the body of second transparent deformable material; and, a posterior member having an anterior side and a posterior side, the anterior side being disposed against the posterior of the body of second transparent deformable material, such that upon application of force against the posterior side of the posterior member with the body of first transparent deformable material restrained so as to cause movement of the posterior member relative to the body of first transparent deformable material, a portion of the second transparent deformable material is extruded through the extrusion aperture so as to form a curved, refractive interface with the body of first transparent deformable material. The intraocular lens is inserted into the posterior chamber of an eye whose natural lens has been removed and whose anterior capsule has been rendered ineffective so that the posterior side of the posterior member is operatively connected to the posterior capsule of the eye. The retaining mechanism is attached to tissue on the interior of the sclera of the eye so as to hold the intraocular lens in place so that when the ciliary muscle is relaxed the posterior capsule pushes the posterior member toward the anterior member causing the second transparent deformable material to be forced into the extrusion aperture so as to form a curved, refractive interface with the first transparent deformable material and, when the ciliary muscle is placed in tension the posterior capsule sags so as to allow the posterior member to move away from the anterior member, to allow the second transparent deformable material to recede from the extrusion aperture and reduce the curvature of the interface.

It is to be understood that this summary is provided as a means for generally determining what follows in the drawings and detailed description, and is not intended to limit the scope of the invention. Objects, features and advantages of the invention will be readily understood upon consideration of the following detailed description taken in conjunction with the accompanying drawings.

### BRIEF DESCRIPTION OF THE DRAWINGS

FIG. 1 is an illustration of the structure and principle of operation of the prior art NuLens IOL with its lens surface curvature corresponding to the natural fully accommodated state of a human eye.

FIG. 2 is an illustration of the NuLens IOL with its lens surface curvature corresponding to the natural fully unaccommodated state of a human eye.

FIG. 3 is an illustration of a general embodiment of a surface curvature accommodating IOL according to the present invention with its lens surface curvature corresponding to the natural fully accommodated state of a human eye.

FIG. 4 is an illustration of the general embodiment of FIG. 3 with its lens surface curvature corresponding to the natural fully unaccommodated state of a human eye.

FIG. 5 is a top view of a first specific embodiment of an accommodating IOL device according to the present invention.

FIG. 6 is a side section of the device of FIG. 5 in its fully compressed state which occurs when the eye is unaccommodated.

FIG. 7 is a side section of the device of FIG. 5 in its fully uncompressed state which occurs when the eye is accommodated.

FIG. 8 is a side section of the device of FIG. 5 in its fully uncompressed state installed in a human eye in its fully accommodated state.

FIG. 9 is a side section of the device of FIG. 5 in its fully compressed state installed in a human eye in its fully unaccommodated state.

FIG. 10 is a side section of a second specific embodiment of an accommodating IOL device according to the present invention in its fully uncompressed state.

FIG. 11 is a side section of the device in FIG. 10 in its fully compressed state.

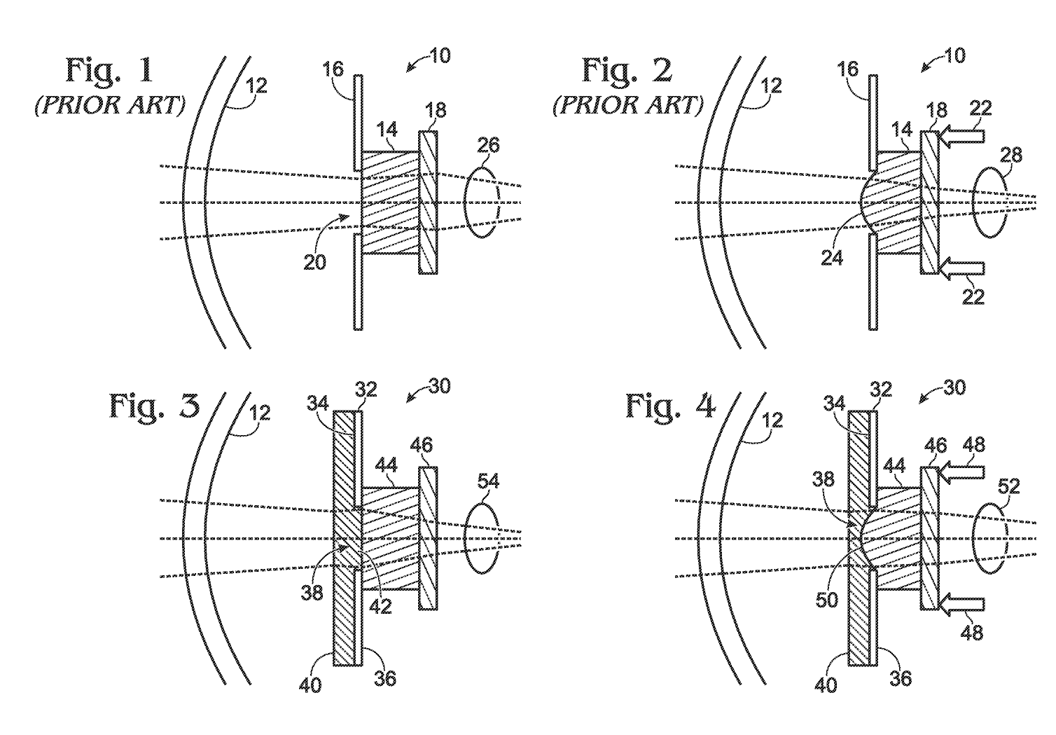

### DETAILED DESCRIPTION OF EMBODIMENTS OF THE INVENTION

The present invention will be readily understood by the following detailed description in conjunction with the accompanying drawings. To facilitate this description, like reference numerals designate like structural elements. In the following description many details are set forth to provide an understanding of the disclosed embodiments of the invention. However, upon reviewing this disclosure, it will become apparent to one skilled in the art that not all of the disclosed details may be required to practice the claimed invention and that alternative embodiments might be constructed without departing from the principles of the invention.

The principle of an accommodating IOL according to the present invention is shown by a general embodiment in FIG. 3, corresponding to the fully accommodated state of the human eye, and FIG. 4, corresponding to the fully unaccommodated state of the human eye. As in the aforementioned prior art, the natural lens of the eye is removed and an accommodating IOL according to the present invention is installed in its place. The anterior portion of the lens capsule is removed or collapsed so that the posterior portion of the IOL rests essentially against the posterior portion of the lens capsule so as to operate the IOL in response to actuation of the ciliary muscles. The lens 30 is illustrated in FIGS. 3 and 4 in relation to the cornea 12 of the eye.

The lens includes a substantially-rigid anterior member 32 having an anterior side 34, a posterior side 36, and an extrusion aperture 38 there through. The anterior member is preferably held in place in the posterior chamber of the eye by a retaining mechanism, as discussed below with respect to specific embodiments of the invention. A body of first transparent deformable material 40, specifically a layer of first transparent elastic material in this example, is disposed on the anterior side of the anterior member. A portion of the layer of first transparent elastic material 42 protrudes into the extrusion aperture. A body of a second transparent deformable material 44, specifically a layer of second transparent elastic material in this example, is disposed on the posterior side of the anterior member covering the extrusion aperture. The second elastic material 44 is harder than the first elastic material and has an index of refraction that is different from the index of refraction of the first elastic material 40. A substantially-rigid posterior member 46 is disposed against the posterior side of the layer of second elastic material 44 over an area corresponding to or larger than the extrusion aperture 38.

As shown in FIG. 4, when a compressive force, shown by arrows 48, is applied to the layer of second transparent elastic material 44 through the posterior plate member 46, the second elastic material partially extrudes through the aperture 38, creating a curved, refractive surface 50 at the interface between the first elastic material 40 and the second elastic material 44. The power of the curved surface can be determined as described above with respect to the prior art, except that the index of refraction on the anterior side of that surface is the index of refraction of the first elastic material rather than the index of refraction of the fluid in the eye.

To overcome the significant problem of the NuLens IOL wherein the accommodated operation of the lens is opposite to that of the natural eye, the index of refraction of the first elastic material is ordinarily higher than the index of refraction of the second elastic material. As in the prior art, when the eye is in its fully unaccommodated state corresponding to FIG. 4, the ciliary muscle dilates and the capsule is stretched. This stretching causes a compressive force 48 to be applied to the posterior member 46. This causes posterior elastic material to be extruded through the aperture 38, forming the curved, refractive surface 50. However, unlike the prior art, because the index of refraction of the first elastic material 40 is higher than the index of refraction of the second elastic material 44, a negative power lens is formed which increases the focal length of the eye as is natural in the unaccommodated state, as shown by rays 52, rather than decreasing the focal length of the eye as in the prior art.

Conversely, when the eye is in its fully accommodated state corresponding to FIG. 3, the ciliary muscle constricts, releasing the tension on the capsule and removing the compressive force on the IOL. The second elastic material 44 retracts from the aperture 38 so that the interface between the first elastic material 40 and the second elastic material 44 flattens, thereby reducing the power of the interface. This decreases the focal length of the eye as is natural in the accommodated state, as shown by rays 54, rather than increasing the focal length of the eye as in the prior art.

For the reasons described above, the index of refraction of the first transparent deformable material 40 would ordinarily be higher than the index of refraction of the second transparent deformable material 44, and the deformability of the second transparent deformable material would be less than the deformability of the first transparent deformable material so that the second deformable material extrudes into the surface of the first deformable material, thereby forming a surface that is convex toward the anterior chamber. However, it is to be understood that other combinations of deformability or hardness and indices of refraction might be used without departing from the principles of the invention. For example, if the hardness of the first elastic material were greater than the harness of the second elastic material and the first elastic material were installed so as to bulge into the second elastic material, the index of refraction of the second elastic material would need to be higher than the index of refraction of the first elastic material to achieve the same ordinary relationship between natural eye accommodation and the state of the accommodating IOL.

In addition, it is to be understood that the first and second elastic layers need not necessarily by monolithic or homogenous to satisfy the principles of the present invention. That is, for example, one or both could actually be a laminate of several different materials to achieve specific mechanical or optical results without departing from the principles of the invention. Further, there may even be circumstances where the index of refraction of the first elastic layer would preferably be lower than the index of refraction of the second elastic layer, with the same relationship of hardness, without departing from the principles of the invention.

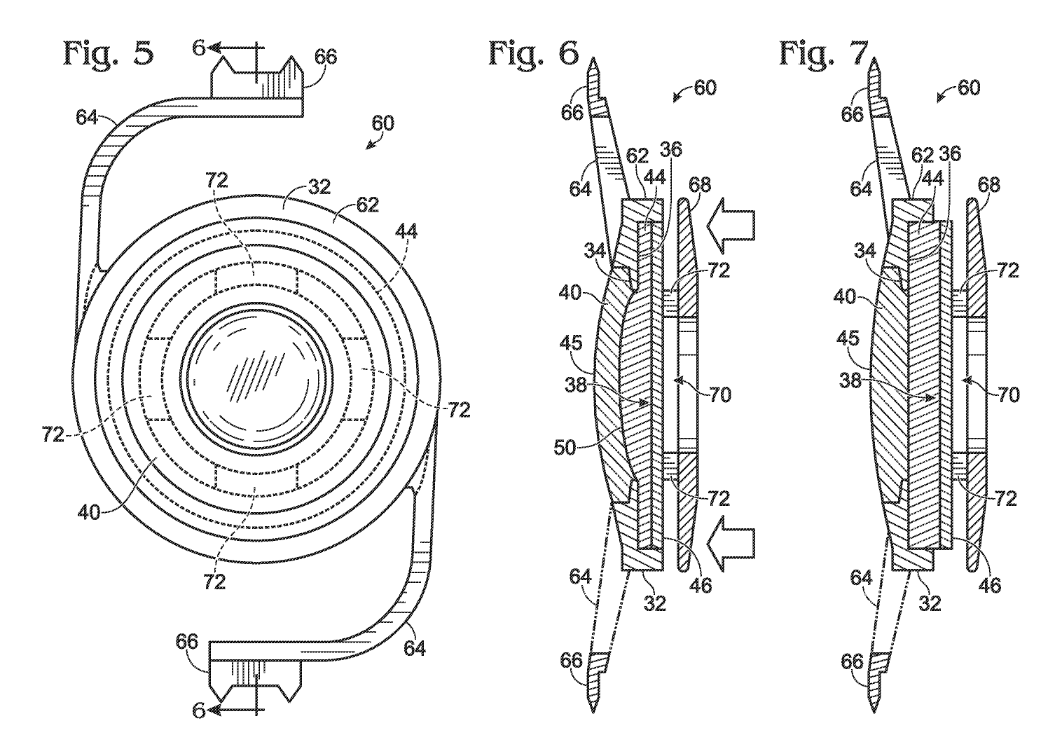

A first specific embodiment of an accommodating IOL device 60 according to the present invention is shown in FIGS. 5, 6 and 7. FIG. 5 is a top view of the device 60. FIG. 6 is a side section of the device 60 in the fully compressed state. FIG. 7 is a side section of the device 60 in the fully uncompressed state. In this embodiment the substantially-rigid anterior member 32 is part of a ring 62 having an anterior inner ledge forming the anterior side 34 on which the first transparent elastic material 40 is mounted and a posterior ledge forming the posterior side 36 on which the second elastic material 44 is mounted. As shown, the anterior surface 45 of the first elastic material may be formed with a curved, powered surface if desired. The posterior member 46 is disposed against the second elastic material 44 within the ring 32.

As is commonly understood in the art relating to IOLs, the device is provided with haptics 64 attached at one end to the ring 62 and having barbs 66 at the other end for securing the IOL device to the interior wall of the eye. To actuate the lens, the device is provided with a posterior annular button 68 having a sight aperture 70 there through and posts 72 for connection to the posterior member 46 to transfer force thereto. The button 68 is adapted to rest against the posterior lens capsule so as to apply force to the posterior member 46 when the eye is in its unaccommodated state and the lens capsule is stretched.

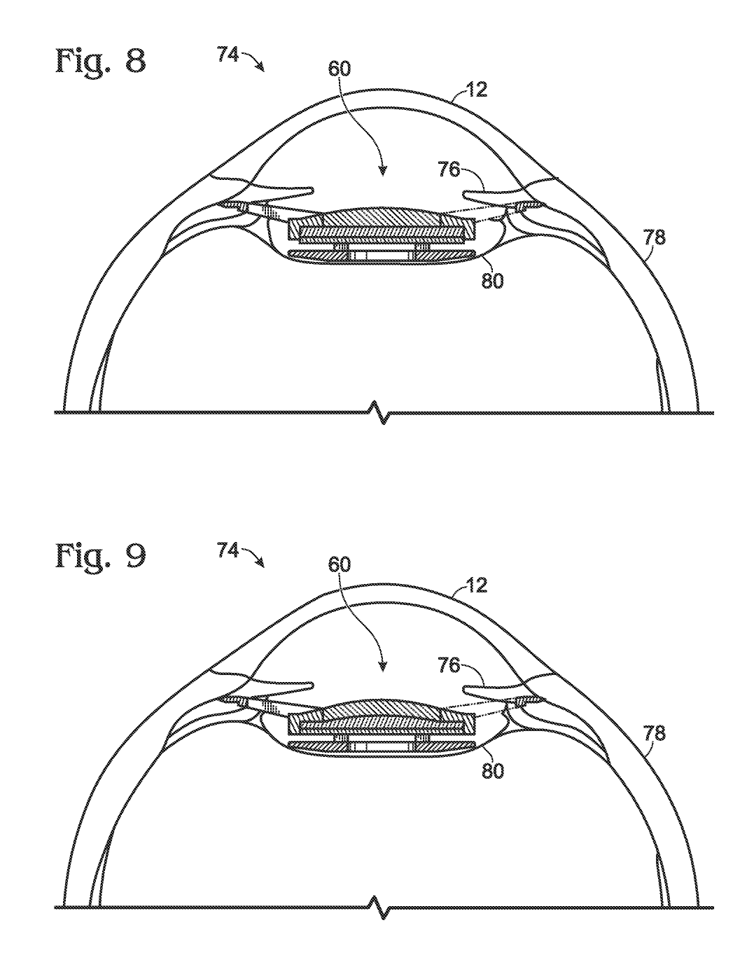

FIG. 9 shows the accommodating IOL embodiment of FIGS. 5, 6 and 7 installed in a human eye 74 having, among the many structures of a human eye, a cornea 12, iris 76, sclera 78 and posterior portion 80 of the lens capsule. In this case, the eye is in its fully unaccommodated state resulting in a stretching of the lens capsule and a compressive force applied to the accommodating IOL. FIG. 8 shows the eye with the IOL installed in the fully accommodating state of the eye resulting in a relaxation of the lens capsule and a reduction or elimination of the compressive force applied to the accommodating IOL.

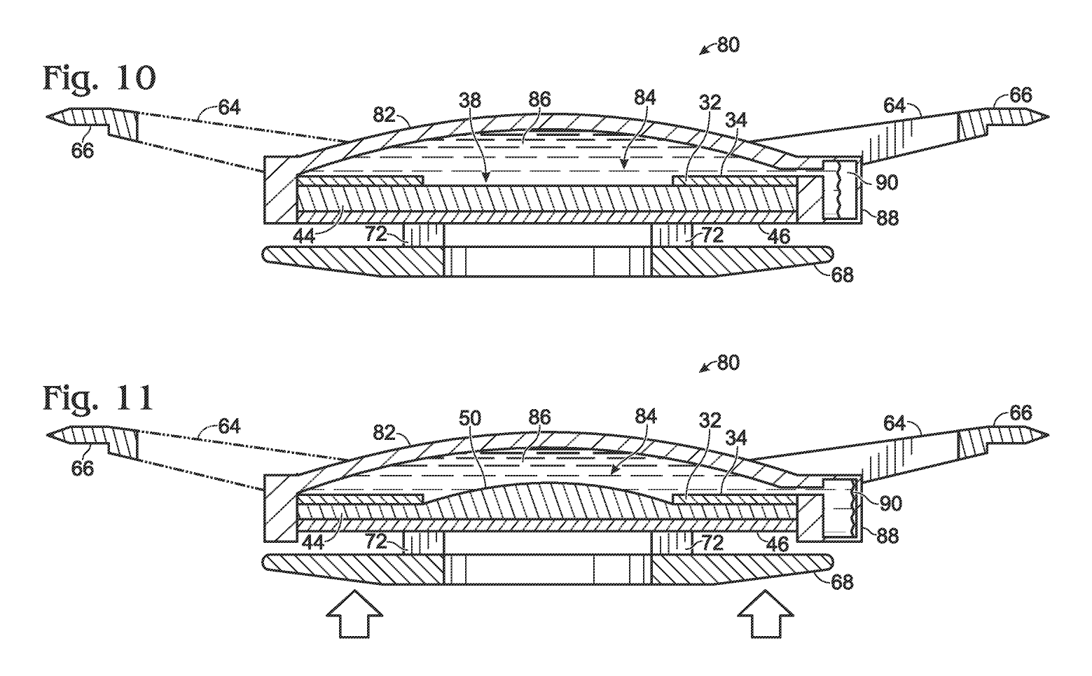

Turning now to FIGS. 10 and 11, a second specific embodiment 80 of an accommodating IOL according to the invention employs a fluid, rather than a solid, as a high index of refraction material on the anterior side of the substantially-rigid anterior member 32. In this case, a preferably domed, transparent cover 82 attached to the substantially-rigid anterior member 32 encloses a space adjacent the anterior side 34 of the anterior member 32 for holding a fluid having an index of refraction that is different, preferably higher, than the index of refraction of the layer of second transparent elastic material 44. Preferably, the fluid is an incompressible fluid 86, in which case a reservoir 88 containing a compressible fluid 90 is included in the cover 82 to allow the incompressible fluid 86 to escape from the space 84 when the layer of second elastic material is extruded through the extrusion aperture 38, as shown specifically in FIG. 11. However, it is to be understood that in some cases it may be desirable to employ only a compressible fluid in the space 84, in which case the reservoir may be unnecessary.

The terms and expressions which have been employed in the foregoing specification are used therein as terms of description and not of limitation, and there is no intention, in the use of such terms and expressions, to exclude equivalents of the features shown and described or portions thereof, it being recognized that the scope of the invention is defined and limited only by the claims that follow.

What is claimed is:

1. An accommodating intraocular lens, comprising:
a substantially-rigid anterior member having an anterior side and a posterior side and an extrusion aperture there through;
a body of a first transparent deformable material disposed anterior to the posterior side of the anterior member at least partially within the periphery of the extrusion aperture;
a body of a second transparent deformable material having an anterior surface disposed at least partially adjacent the posterior surface of the body of first transparent material, the second transparent deformable material having a different degree of deformability than the first transparent deformable material and having an index of refraction different from the index of refraction of the first transparent deformable material, thereby forming a refractive deformable interface between the body of first transparent deformable material and the body of second transparent deformable material; and
a posterior member having an anterior side and a posterior side, the anterior side being disposed against the posterior of the body of second transparent deformable material, such that upon application of force against the posterior side of the posterior member with the body of first transparent deformable material restrained so as to cause movement of the posterior member relative to the body of first transparent deformable material, a portion of the second transparent deformable material is extruded through the extrusion aperture so as to form a curved, refractive interface with the body of first transparent deformable material.

2. The accommodating intraocular lens of claim 1, wherein the body of a first transparent deformable material comprises a layer of a first elastic material disposed on the anterior side of the anterior member and having a portion thereof protruding into the extrusion aperture; and the body of a second transparent deformable material comprises a layer of a second elastic material disposed on the posterior side of the anterior member covering the extrusion aperture, the second elastic material being harder than the first elastic material.

3. The lens of claim 2, further comprising a haptic connected to the anterior member for holding the anterior member in the posterior chamber of the eye.

4. The lens of claim 3, wherein the index of refraction of the first elastic material is higher than the index of refraction of the second elastic material.

5. The lens of claim 2, wherein the index of refraction of the first elastic material is higher than the index of refraction of the second elastic material.

6. The lens of claim 2, wherein the shape of the extrusion aperture is adapted to control the shape of the curved refractive interface between the first and second elastic materials.

7. The lens of claim 6, wherein the shape of the extrusion aperture is elliptical to provide a curved refractive interface between the first and second elastic materials wherein the curvatures of the refractive interface along the major and minor axes of the ellipse are different.

8. The accommodating intraocular lens of claim 2, wherein the first elastic material is disposed at least partially on the anterior side of the anterior member, and the second elastic material is disposed at least partially on the posterior side of the anterior member.

9. The accommodating intraocular lens of claim 1, wherein the body of a first transparent deformable material comprises a transparent fluid, and the lens further comprises an at least partially transparent chamber disposed on the anterior side of the anterior member for holding the fluid and bound by the anterior side of the anterior member and the extrusion aperture.

10. The accommodating intraocular lens of claim 9, wherein the fluid is an incompressible fluid and the lens further comprises a reservoir to hold excess fluid from the chamber.

11. The accommodating intraocular lens of claim 9, wherein the fluid is a compressible fluid.

12. The lens of claim 10, wherein the index of refraction of the fluid is higher than the index of refraction of the second transparent deformable material.

13. The lens of claim 9, wherein the body of a second transparent deformable material comprises a layer of elastic material.

14. The accommodating intraocular lens of claims 13, wherein the elastic material is disposed at least partially in the posterior side of the interior member.

15. The lens of claim 13, wherein the index of refraction of the fluid is higher than the index of refraction of the elastic material.

16. The lens of claim 15, wherein the shape of the extrusion aperture is adapted to control the shape of the curved refractive interface between the fluid and the elastic material.

17. The lens of claim 15, wherein the shape of the extrusion aperture is elliptical to provide a curved refractive interface between the fluid and the elastic material wherein the curvatures of the refractive interface along the major and minor axes of the ellipse are different.

18. The lens of claim 9, wherein the shape of the extrusion aperture is adapted to control the shape of the curved refractive interface between the fluid and the elastic material.

19. The lens of claim 18, wherein the shape of the extrusion aperture is elliptical to provide a curved refractive interface between the fluid and elastic material where the curvatures of the refractive interface along the major and minor axes of the ellipse are different.

20. A method for providing an eye with an accommodating intraocular lens, comprising:
providing an accommodating intraocular lens having:
a substantially-rigid anterior member having an anterior side and a posterior side and an extrusion aperture there through;
a body of a first transparent deformable material disposed anterior to the posterior side of the anterior member at least partially within the periphery of the extrusion aperture;
a body of a second transparent deformable material having an anterior surface disposed at least partially adjacent the posterior surface of the body of first transparent material, the second transparent deformable material having a different degree of deformability than the first transparent deformable material and having an index of refraction different from the index of refraction of the first transparent deformable material, thereby forming a refractive deformable interface between the body of first transparent deformable material and the body of second transparent deformable material; and
a posterior member having an anterior side and a posterior side, the anterior side being disposed against the posterior of the body of second transparent deformable material, such that upon application of force against the posterior side of the posterior member with the body of first transparent deformable material restrained so as to cause movement of the posterior member relative to the body of first transparent deformable material, a portion of the second transparent deformable material is extruded through the extrusion aperture so as to form a curved, refractive interface with the body of first transparent deformable material;
inserting the intraocular lens into the posterior chamber of an eye whose natural lens has been removed and whose anterior capsule has been rendered ineffective so that the posterior side of the posterior member is operatively connected to the posterior capsule of the eye; and
attaching the retaining mechanism to tissue on the interior of the sclera of the eye so as to hold the intraocular lens in place so that when the ciliary muscle is relaxed the posterior capsule pushes the posterior member toward the anterior member causing the second transparent deformable material to be forced into the extrusion aperture so as to form a curved, refractive interface with the first transparent deformable material and, when the ciliary muscle is placed in tension the posterior capsule sags so as to allow the posterior member to move away from the anterior member, to allow the second transparent deformable material to recede from the extrusion aperture and reduce the curvature of the interface.

21. The method of claim 20, further comprising providing the intraocular lens with a haptic connected to the anterior member for holding the anterior member in the posterior chamber of the eye.

22. The method of claim 21, further comprising providing the intraocular lens with a first deformable material whose index of refraction is higher than the index of refraction of the second deformable material.

23. The method of claim 20, further comprising providing the intraocular lens with a first deformable material whose index of refraction is higher than the index of refraction of the second deformable material.

24. The method of claim 20, wherein providing an accommodating intraocular lens having a body of a first transparent deformable material and a body of a second transparent deformable material comprises providing as the first transparent deformable material a layer of first elastic material disposed at least partially on the anterior side of the anterior member and having a portion thereof protruding into the extrusion member, and a layer of second elastic material disposed at least partially on the posterior side of the anterior member, the second transparent elastic material being harder than the first transparent elastic material.

25. The method of claim 24, further comprising providing the intraocular lens with a haptic connected to the anterior member for holding the anterior member in the posterior chamber of the eye.

26. The method of claim 24, further comprising providing the intraocular lens with a first elastic material whose index of refraction is higher than the index of refraction of the second elastic material.

27. The method of claim 26, further comprising providing the intraocular lens with a haptic connected to the anterior member for holding the anterior member in the posterior chamber of the eye.

28. The method of claim 20, wherein providing an accommodating intraocular lens having a body of a first transparent deformable material and a body of a second transparent deformable material comprises providing as the first transparent deformable material a transparent fluid in an at least partially transparent chamber disposed on the anterior side of the anterior member for holding the fluid and bound by the anterior side of the anterior member and the extrusion aperture.

29. The method of claim 28, wherein providing a transparent fluid in a transparent chamber includes providing an incompressible fluid and further comprises providing the chamber with a reservoir for receiving excess fluid.

30. The method of claim 28, wherein providing a transparent fluid in a transparent chamber includes providing a compressible fluid.

31. The method of claim 28, wherein providing an accommodating intraocular lens having a body of a second transparent deformable material having an anterior surface disposed adjacent the posterior surface of the body of first transparent material comprises providing a layer of elastic material disposed on the posterior side of the anterior member.

32. The method of claim 31, further comprising providing the intraocular lens with a haptic connected to the anterior member for holding the anterior member in the posterior chamber of the eye.

33. The method of claim 31, further comprising providing the intraocular lens with a fluid whose index of refraction is higher than the index of refraction of the layer of elastic material.

34. The method of claim 33, further comprising providing the intraocular lens with a haptic connected to the anterior member for holding the anterior member in the posterior chamber of the eye.
